(19) United States
(12) Patent Application Publication
(10) Pub. No.: US 2004/0067934 A1
(43) Pub. Date: Apr. 8, 2004

(54) Use of galantamine for the treatment of neuropsychiatric behaviour associated with alzheimer's disease

(76) Inventors: Wim Louis Julien Parys, Gaithersburg, MD (US); Michael Pontecorvo, Lawrenceville, NJ (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/263,876

(22) Filed: Oct. 3, 2002

(51) Int. Cl. A61K 31/55

(52) U.S. Cl. 514/214.03

(57) ABSTRACT

Galantamine has be used in the treatment of a number of chronic diseases. Galantamine has been found to be safe and effective in the treatment of Alzheimer's disease. Neuropsychiatric disorders are often associated with Alzheimer's disease. It is demonstrated that galantamine is also effective in reducing or stabilizing the incidence of neuropsychiatric behaviour seen in Alzheimer's patients.

The present invention relates to the use of an effective amount of galantamine for the production of a medicament for the treatment of neuropsychiatric behaviour associated with Alzheimer's disease.

### BACKGROUND OF THE INVENTION

Galantamine is a reversible cholinesterase inhibitor that can be isolated from a number of different plant sources, including daffodil bulbs. Galantamine interacts competitively with the enzyme, acetylcholinesterase, and demonstrates a 10 to 50 fold selectivity for acetyl vs. butyryl cholinesterase.

Galantamine has been used for the treatment of a number of chronic diseases, where life-long treatment may be necessary. Galantamine has been shown to be effective in the treatment of arthritic disorders (Canadian Patent application 2,251,114); fatigue syndromes (Canadian Patent application 2,108,880); mania (Canadian Patent application 2,062,094); schizophrenia (Canadian Patent application 2,108,880); memory dysfunction, including Alzheimer's Disease (U.S. Pat. No. 4,663,318); alcoholism (Canadian Patent 2,039,197); nicotine dependence (Canadian Patent application 2,153,570); disorders of attention (PCT publication WO 99/21561) and jet lag (Canadian Patent application 2,193,473).

However, none of the studies demonstrate the usefulness of galantamine for the treatment of neuropsychiatric behaviour associated with Alzheimer's disease.

### SUMMARY OF THE INVENTION

Thus, according to the present invention there is provided a use of of galantamine for the production of a medicament for the treatment of neuropsychiatric behaviour associated with Alzheimer's disease.

In a further embodiment there is provided a method of treating neuropsychiatric behaviour associated with Alzheimer's disease by administering to a patient in need thereof a safe and effective dose of galanatamine or a pharmaceutically acceptable salt thereof.

### BRIEF DESCRIPTION OF THE DRAWINGS

These and other features of the invention will become more apparent from the following description in which reference is made to the appended drawings wherein:

FIG. 1 shows the mean change from baseline by treatment group over time in ADAS-cog/11 (observed case).

FIG. 2 shows the mean change from baseline by treatment group over time in CIBIC-plus (observed case).

FIG. 3 shows the cumulative percentage of patients with specified changed from baseline at Month 5 in ADAS-cog/11 scores.

FIG. 4 shows the change in ADL performance from baseline over time at Month 5.

FIG. 5 shows the change in NPI score from baseline over time to Month 5.

### DESCRIPTION OF PREFERRED EMBODIMENT

The present invention relates to the use of an effective amount of galantamine for the treatment of neuropsychiatric behaviour associated with Alzheimer's disease.

Galantamine, a tertiary alkaloid, has been isolated form the bulbs of the Caucasian snowdrops Galantanus woronowi (Proskurnina, N. F. and Yakoleva, A. P. 1952, Alkaloids of Galanthus woronowi. II. Isolation of a new alkaloid. (In Russian.) Zh.Obschchei Khim. (J. Gen. Chem.) 22, 1899-1902). It has also been isolated from the common snowdrop Galanthus nivalis (Boit, 1954). Galantamine is a well-known acetylcholinesterase inhibitor which is active at nicotinic receptor sites but not on muscarinic receptor sites. It is capable of passing the blood-brain barrier in humans, and presents no severe side effects in therapeutically effective dosages.

Galantamine has been used extensively as a curare reversal agent in anaesthetic practice in Eastern bloc countries (cf. review by Paskow, 1986) and also experimentally in the West (cf. Bretagne and Valetta, 1965: Wislicki, 1967; Consanitis. 1971).

Galantamine has been marketed by the company Waldheim (Sanochemia Gruppe) as Nivalin™ in Germany and Austria since the 1970s for indications such as facial neuralgia.

In the present invention when we refer to galantamine we include within this term galantamine itself, derivatives and salts thereof, such as halides, for example galantamine hydrobromide.

For the purposes of the present invention galantamine and derivates and salts thereof may be formulated according to convention methods of pharmacy, together where appropriate with one or more pharmaceutically acceptable carriers, excipients or diluents, as is known in the art. Such formulations can take the form of tablets, capsules, solutions, or lozenges, pessaries, creams, suppositories or transdermal formulations, depending on the route of administration.

Galantamine has been used for the treatment of a number of chronic diseases, where life-long treatment may be necessary. Galantamine has been shown to be effective in the treatment of arthritic disorders (Canadian Patent application 2,251,114); fatigue syndromes (Canadian Patent application 2,108,880); mania (Canadian Patent application 2,062,094); schizophrenia (Canadian Patent application 2,108,880); memory dysfunction, including Alzheimer's Disease (U.S. Pat. No. 4,663,318); alcoholism (Canadian Patent 2,039,197); nicotine dependence (Canadian Patent application 2,153,570); disorders of attention (PCT publication WO 99/21561) and jet lag (Canadian Patent application 2,193,473).

According to the present invention a safe and effective amount of galantamine can be used for the treatment of neuropsychiatric behaviour associated with Alzheimer's disease.

Precise dosage rates and regimes can be determined empirically by the medical practitioner, depending on individual circumstances. For example, if the compound is delivered orally, a daily dose of about 1 mg to about 100 mg. In a further example the compound can be delivered at about 5 mg to about 50 mg per day. In yet a further example the compound can be delivered at about 16 mg to about 32 mg per day. Precise daily dosages can be selected from 16 mg, 18 mg, 24 mg or 32 mg per day. It is preferred that the daily dosage be divided into two or three equal dosages.

In one embodiment of the present invention it has been found that the tolerability or safety of the drug can be improved if the patient is introduced to the drug slowly over a number of weeks.

In one embodiment of the present invention the patient is introduced to galantamine slowly from about 2 weeks to about 10 weeks, wherein the dose is increased over this period.

In one embodiment of the present invention the patient receives a dose of about 8 mg/day for about 1 week, followed by a dose of about 16 mg/day for about 1 week, followed by a maintenance dose of about 24 mg/day thereafter.

In one embodiment of the present invention the patient receives a dose of about 8 mg/day for about 1 week, followed by a dose of about 16 mg/day for about 1 week, followed by a dose of about 24 mg/day for about a week, followed by a maintenance dose of about 32 mg/day thereafter.

In one embodiment of the present invention the patient receives a dose of about 8 mg/day for from about 2 weeks to about 4 weeks, followed by a dose of about 16 mg/day for from about 2 weeks to about 4 weeks, followed by a maintenance dose of about 24 mg/day thereafter.

In one example of this embodiment the patient receives a dose of about 8 mg/day for about 4 weeks, followed by a dose of about 16 mg/day for about 4 weeks, followed by a maintenance dose of about 24 mg/day thereafter.

In a further embodiment of the present invention the patient receives a dose of about 8 mg/day for from about 2 weeks to about 4 weeks, followed by a maintenance dose of about 16 mg/day thereafter. In one example of this embodiment the patient receives a dose of about 8 mg/day for about 4 weeks, followed by a maintenance dose of about 16 mg/day thereafter.

According to the present invention, the neuropsychiatric behaviour associated with Alzheimer's Disease includes for example: delusions, hallucinations, agitation/aggression, dysphoria, anxiety, euphoria, apathy, disinhibition, irritability/lability and aberrant motor behavior.

The present invention is illustrated by the following example, which is not to be construed as limiting.

### EXAMPLES

Patients diagnosed with Alzheimer's Disease (approximately 910) were randomized to one of four treatment arms: placebo; 8 weeks titration to galantamine 24 mg/day; 4 weeks titration to galantamine 16 mg/day, or galantamine 8 mg/day, no titration needed, for five months. Patients included in this study must have been diagnosed with Alzheimer's Disease, had an Alzheimer's Disease Assessment Scale (Rosen, W. G. et al., Amer. J. Psychiatry, 141: 1356-1364, 1984) cognitive portion (ADAS-cog-11) score of at least 18 and had a history of cognitive decline that was gradual at the onset and progressive over a period of at least six months.

The titration schedules for the various treatment arms are as follows:

Subjects in the Placebo group received 21 weeks (5 months) of placebo medication. Subjects in group Gal 24 received 4 weeks of 8 mg/day galantamine (4 mg, twice daily (bid)), 4 weeks of 16-mg/day galantamine (8 mg, bid) and 13 weeks of 24 mg/day galantamine (12 mg, bid). Subjects in group Gal 16 received 4 weeks of 8 mg/day galantamine (4 mg, bid) and 17 weeks of 16-mg/day galantamine (8 mg, bid). Subjects in group Gal 8 received 8 mg/day (4 mg, bid) immediately upon randomization and continued on that dose for 21 weeks.

All patients were monitored throughout the study, with follow-up and cognitive evaluation at four weeks, three months and five months after the start of the study.

The primary efficacy endpoints were the change from baseline ADAS-cog/11 and the CIBIC-plus score (Clinician's Interview Based Impression of Change Plus Family Input) at month five. These two tests together with the Mini-Mental State Examination (MMSE), which was performed at the screening stage, are discussed below:

The ADAS consists of two parts—a cognitive subscale and a behavioral subscale. The behavioral subscale was not be used in this study. The cognitive subscale, the ADAS--cog-11, consisted of Word Recall and Word Recognition memory tests, Object and Finger Naming, Commands, Constructional Praxis, Ideational Praxis, Orientation, Remembering Test Instructions, Spoken language Ability, Comprehension of Spoken language and Word Finding Difficulty was the primary variable in this study.

In addition to the above specified items from the ADAS-cog-11, two additional ADAS items were assessed: The Concentration and Distractibility item, originally part of the behavioral subscale, was performed and a Delayed Word Recall test (delayed recall of the word recall items) was added to give additional information regarding cognitive status. The expanded 13 item ADAS (ADAS-cog 13) was a secondary variable.

To reduce variability due to circadian fluctuations in cognitive status the ADAS was done always at the same time of the day, preferably before noon. Only a trained ADAS rater performed the test. Ideally the ADAS rater was not involved in the treatment of the subject and should have no access to AE (adverse event) reporting.

The ADAS was performed at visits 1, 2, 3, 4 and 5 (screening, baseline, week 4, week 13 and month 5 or upon early discontinuation of trial medication intake). For word recall and word recognition two parallel wordlists, list A and list B were employed. List A was used at visits 1 and 3, List B at visits 2, 4, and 5 or upon early discontinuation of trial medication intake. For practical reasons the words for word recognition was presented only once. The total score of the 11 cognitive items on the original ADAS cognitive subscale (ADAS-cog/11, Range: 0-70) was recorded.

The CIBIC-plus score was a second primary variable. An independent, experienced and properly trained clinician provided a global impression of the subject's deterioration or improvement over the course of the trial, based on separate interviews with the subject and caregivers. If helpful, the CIBIC rater audiotaped or videotaped the baseline interview for future reference.

Change from baseline was rated on an 7 point scale, where 1 indicates markedly improved, 4 indicates no change and 7 indicates markedly worse. The CIBIC-plus was performed at visit 2, 3, 4, and 5 (baseline, week 4, week 13, and month 5 or upon early discontinuation of trial medication intake). Only a trained CIBIC rater performed the test.

The MMSE is a very brief test of cognitive functions including orientation to time and place, instantaneous recall, short-term memory, and ability to perform serial subtractions or reverse spelling, constructional capacities and the use of language. The MMSE score was derived from the sum of the points assigned to each completed task. A total possible score is 30. The MMSE will be performed at visit 1 (screening).

Secondary efficacy variables include ADAS-cog/11 and the ADCS/ADL scale. The ADCS/ADL test is discussed below:

The ADCS/ADL scale is a 23-item informant-based assessment scale measuring widely applicable daily activities appropriate for patients in the mild to moderate category of Alzheimer's Disease. The 23 items were selected for measurement from the larger set of 45 items studied by Galasko et al (Alzheimer Disease and Associated Disorders, Vol 11, Suppl. 2, 1997). These individual items were scored from 0-3 to 07, depending on the question, with a possible total score of 78. A higher score indicated a higher functioning patient.

The items and scoring were as follows:

Eating (0-3)

Walking (0-3)

Toileting (0-3)

Bathing (0-3)

Grooming (0-3)

Dressing

selection of clothes (0-3)

physical performance (0-4)

Telephone (0-5)

Television (0-3)

Conversation (0-3)

Dishes (0-3)

Managing personal belongings (0-3)

Obtaining beverages (0-3)

Making a meal or snack (0-4)

Disposal of garbage (0-3)

Travel outside home (0-4)

Shopping (0-4)

Keeping appointments (0-3)

Ability to be left alone (0-3)

Current events (0-3)

Reading (0-2)

Writing (0-3)

Hobbies (0-3)

Household appliances (0-4)

Neuropsychiatric behavior was monitored by a test known as The Neuropsychiatric Inventory (NPI) (Cummings, J. L. et al., Neurology, 44: 2308-2314, 1994). The NPI covers 10 domains of behaviors reported in patients with Alzheimer's Disease: delusions, hallucinations, agitation/aggression, dysphoria, anxiety, euphoria, apathy, disinhibition, irritability/lability and aberrant motor behavior. For each domain abnormal behavior can be absent (score 0) or present. If present, the frequency and severity of abnormal behavior is rated based on answers to a set of subquestions regarding behaviors relevant to that domain. Severity was rated 1 to 3 as mild, moderate or marked. Frequency was rated 1 to 4 as occasionally, often, frequently and very frequently. The product of frequency and severity (maximum score=12) was calculated for each domain. A total of NPI was calculated as the sum of the frequency and severity products (maximum score=120). The NPI was performed at visits 2, 3, 4 and 5 (baseline, week 4, week 13, and month 5 or upon early discontinuation of trial medication).

All data was compared among the treatment groups—placebo, galantamine 8 mg/day, 16 mg/day and 24 mg/day.

Between treatment groups comparisons (with particular focus on differences from placebo) were done at each scheduled time interval and for each endpoint imputation scheme. These comparisons will be based on the change from baseline scores for efficacy parameters with baseline (e.g., ADAS-cog/11) and the original scored for efficacy parameters without baseline e.g., CIBIC-plus).

For continuous data, a two-way analysis of variance (ANOVA) model with treatment and investigator as factors were used to compare the treatment groups for the change from baseline data. The interaction between treatment and investigator was examined. The impact of the baseline score on change from baseline was evaluated. If the baseline score was found to be a relevant predictor (p<10), an analysis of covariance model (ANCOVA) was used to assess the treatment effects and the interaction between treatment and baseline score was examined. If the parametric methods were deemed inappropriate (normality assumption violated), nonparametric methods such as two-way ANOVA on ranked data, Van Elteren test, controlling for investigator, was used. Following ANOVA, Fisher's LSD procedure was used for pairwise comparisons between each galanthamine group and the placebo group. A linear contrast on the main effect for treatment was used to test the dose response relationship.

For ordinal categorical variables such as the CIBIC-plus score, the Van Elteren test controlling for investigator was used for the between group comparison. For the nominal data (e.g., events rates), the Cochran-Mantel-Haenszel test for general association controlling for investigator was used. A linear contrast on the proportion of patients that stay the same or improve was used to test for increasing response with increasing dose.

If a significant proportion of subjects discontinue prematurely, additional analyses were preformed to evaluate the impact on the results. In addition to the by-visit analysis, method for analysing continuous repeated measures were used to evaluate the treatment effect over time.

The safety of the drug was also monitored throughout the study. Blood samples for biochemistry and haematology and random urine sample for urinalysis were taken at each visit and at completion for all efficacy testing. Systolic and diastolic blood pressure were measured in the sitting position, pulse and vital signs were recorded at each visit.

Patient Demographics and baseline characteristics were to be well balanced across all treatment groups (Table 1). The baseline cognitive performance for these Alzheimer's disease patients was mild to moderate as measured by the MMSE and ADAS-cog/11 scores of approximately 18 and 28 to 20.

TABLE 1
Demographics and baseline characteristics

| Trial disposition and | | | | | | | | |
|---|---|---|---|---|---|---|---|---|
| patient characteristics | Placebo | | GAL 8 mg | | GAL 16 mg | | GAL 24 mg | |
| Total Number of Patients | 286 | | 140 | | 279 | | 273 | |
| Completed: N (%) | 240 | (84%) | 108 | (77%) | 219 | (79%) | 212 | (78%) |
| Gender | | | | | | | | |
| Male | 108 | (38%) | 50 | (36%) | 105 | (38%) | 90 | (33%) |
| Female | 178 | (62%) | 90 | (64%) | 174 | (62%) | 183 | (67%) |
| Age: (Years) | | | | | | | | |
| Mean (SE) | 77.1 | (0.46) | 76.0 | (0.61) | 76.3 | (0.49) | 77.7 | (0.43) |
| Median (Min-Max) | 78 | (53-100) | 77 | (52-91) | 77 | (51-94) | 78 | (57-95) |
| Race | | | | | | | | |
| Black | 13 | | 5 | | 12 | | 14 | |
| Caucasian | 267 | (93%) | 132 | (94%) | 260 | (93%) | 249 | (91%) |
| Hispanic | 3 | | 3 | | 5 | | 4 | |
| Oriental | 3 | | 0 | | 1 | | 3 | |
| Other | 0 | | 0 | | 1 | | 3 | |
| Sum of MMSE: | | | | | | | | |
| Mean (SE) | 17.7 | (0.21) | 18.0 | (0.30) | 17.8 | (0.21) | 17.7 | (0.23) |
| Median (Min-Max) | 19 | (10-22) | 19.0 | (10-22) | 19 | (10-22) | 19.0 | (10-22) |
| Baseline ADAS-cog/11 | | | | | | | | |
| Mean (SE) | 29.4 | (0.63) | 27.8 | (0.94) | 29.4 | (0.66) | 29.0 | (0.67) |
| Median (Min-Max) | 27 | (10-61) | 26 | (11-62) | 28 | (10-62) | 27 | (10-54) |

The number of patients randomized among the four treatment groups was 978. The total number of patients completing this trail was high (approximately 80%) with a relatively even rate of discontinuation due to adverse events was relatively infrequent and evenly distributed among all treatment groups (see Table 2).

TABLE 2
Discontinuation of trial medication

| Trial | | GAL | GAL | GAL |
|---|---|---|---|---|
| termination reasons | Placebo | 8 mg/day | 16 mg/day | 24 mg/day |
| Total patients | 286 | 140 | 279 | 273 |
| Total completed: | 240 (84%) | 108 (77%) | 219 (79%) | 212 (78%) |
| N (%) | | | | |
| Total discontinued | 46 (16%) | 32 (23%) | 60 (22%) | 61 (22%) |
| (DC): N (%) | | | | |
| DC due to adverse | 20 (7%) | 9 (6%) | 19 (7%) | 27 (10%) |
| event | | | | |
| DC due to inefficacy | 0 | 1 (1%) | 0 | 2 (1%) |
| DC due to other^{a} | 23 (8%) | 18 (13%) | 29 (10%) | 20 (7%) |
| DC due to ineligible to | 0 | 0 | 4 (1%) | 2 (1%) |
| continue | | | | |
| DC due to non- | 3 (1%) | 4 (3%) | 7 (3%) | 10 (4%) |
| compliance | | | | |
| DC due to withdrawal | 0 | 0 | 1 (0.4%) | 0 |
| of consent | | | | |

^{a}The majority of discontinuations due to other reasons were for withdrawal of consent.

In this study there were two primary efficacy endpoints according to widely used international standards: change in ADAS-cog/11 score at Month 5 compared to baseline and CIBIC-plus score at Month 5.

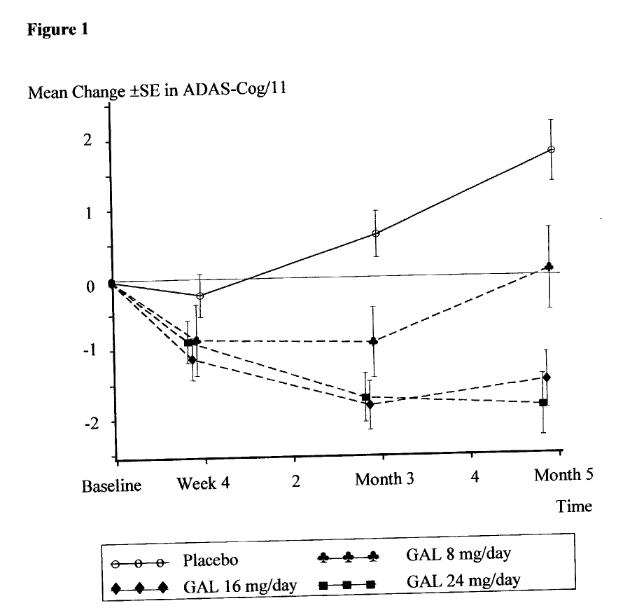

As shown in Table 3 and FIG. 1, a statistically significant treatment effect was shown for the 16 and 24 mg/day galantamine treatment groups compared with placebo for the ADAS-cog/11. Results from analysis based on the last observation carried-forward (LOCF) data corroborate the result based on observed data. The 8 mg/day galantamine group was significantly different from placebo for the observed case but not for the LOCF. Galantamine at a dose of 24 mg/day did not appear to be significantly more effective than 16 mg/day. However, the duration of exposure to the target dose differed by 1 month between the two treatment groups (two months versus three months respectively).

TABLE 3
Change from baseline in ADAS-cog/11 at Month 5

| | Placebo | GAL 8 mg | GAL 16 mg | GAL 24 mg |
|---|---|---|---|---|
| Month | n = 225 | n = 101 | n = 208 | n = 211 |
| 5: (ob- | | | | |
| served | | | | |
| case) | | | | |
| Mean | 1.8 (0.43) | 0.1 (0.58)* | −1.5 (0.40)***^{†} | −1.8 (0.44)***^{†} |
| (SE) | | | | |
| Month | n = 255 | n = 126 | n = 253 | n = 253 |
| 5: | | | | |
| (LOCF) | | | | |
| Mean | 1.7 (0.39) | 0.4 (0.52)♦ | −1.4 (0.35)***^{‡} | −1.4 (0.39)***^{‡} |
| (SE) | | | | |

Lower score indicates better condition. P-Values based on two-way ANOVA model.
Significantly more effective than placebo: *p ≦ 0.05; **p ≦ 0.01; ***p ≦ 0.001;
Approached significance: ♦0.05 < p-value < 0.10.
Significantly more effective than 8 mg/day: ^{†}p ≦ 0.05; ^{‡}p ≦ 0.01.

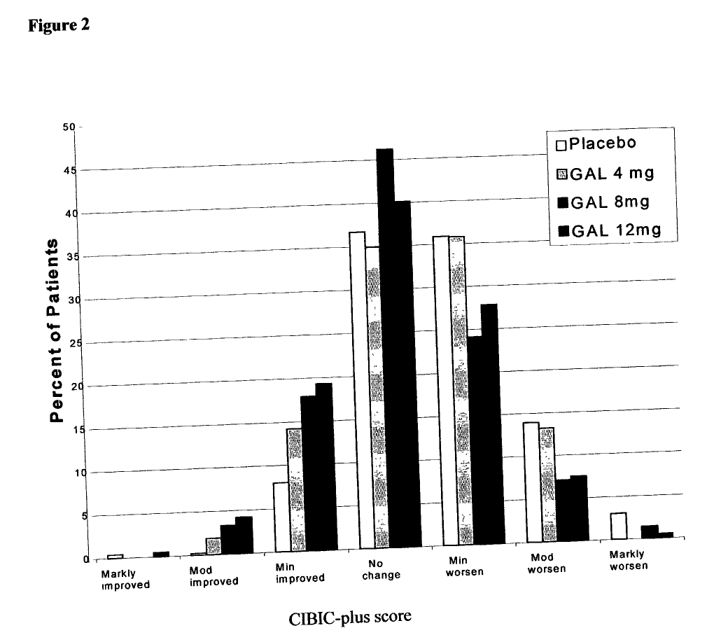

For the CIBIC-plus assessment at Month 5, the percent of patients with improved or unchanged scores was significantly greater with galantamine treatment with 16 or 24 mg/day compared with placebo or 8 mg/day of galantamine (Table 4). After 5 months of treatment, 64% to 68% of patients with 24 or 16 mg/day of galantamine showed improvement or were unchanged from baseline compared with 47% to 51% with placebo or 8 mg/day of galantamine. The analysis of imputed data at LOCF endpoint gave similar results. There was an apparent dose-related increase in the percentage of patients showing improvement or no change in the CIBIC-plus (FIG. 2).

TABLE 4
CIBIC-plus at Month 5 for improved or unchanged scores

| | Placebo | GAL 8 mg | GAL 16 mg | GAL 24 mg |
|---|---|---|---|---|
| Month 5: | n = 237 | n = 106 | n = 212 | n = 212 |
| (observed | | | | |
| case) | | | | |
| Improved/ | 112 (47%) | 54 (51%) | 143 (68%)***^{†} | 136 (64%)***^{‡} |
| no change | | | | |
| n (%) | | | | |
| Month 5: | n = 263 | n = 128 | n = 255 | n = 253 |
| (LOCF) | | | | |
| Improved/ | 128 (49%) | 68 (53%) | 169 (66%)***^{†} | 162 (64%)***^{‡} |
| no change | | | | |
| n (%) | | | | |

P-value from Van Elteren test on the 7-point scale
Significantly more effective than placebo: *p ≦ 0.05; **p ≦ 0.01; ***p ≦ 0.001.
Significantly more effective than 8 mg/day: ^{†}p ≦ 0.05; ^{‡}p ≦ 0.01.

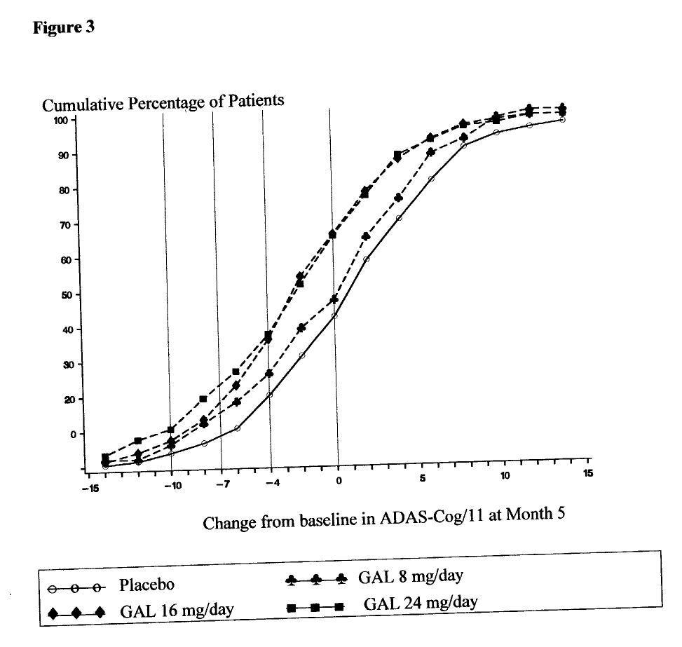

At Month 5 there were significantly more patients who responded with no change or improved scores with 16 and 24 mg/day of galantamine compared with placebo or 8 mg/day of galantamine. Patients responding with ADAS-cog/11 changes from baseline of 7 or more points occurred in 15.9% and 22.3% of patients in the 16 and 24 mg/day groups, respectively, compared with the placebo group (7.6%). There was overall a higher cumulative percentage of patients with galantamine treatment who responded with a minimum improvement of any magnitude compared with placebo (FIG. 3).

TABLE 5
Responders analysis based on change in
ADAS-cog/11 score from baseline at Month 5

| Definition | | | | |
|---|---|---|---|---|
| of | | | | |
| responder | Placebo | GAL 8 mg | GAL 16 mg | GAL 24 mg |
| | n = 225 | n = 101 | n = 208 | n = 211 |
| Change ≦ | 94 (41.8) | 47 (46.5) | 136 (65.4)***^{‡} | 137 (64.9)***^{‡} |
| 0 points | | | | |
| n (%) | | | | |
| Change ≦ | 44 (19.6) | 26 (25.7) | 74 (35.6)***♦ | 78 (37.0)***^{†} |
| −4 points | | | | |
| n (%) | | | | |
| Change ≦ | 17 (7.6) | 14 (13.9) | 33 (15.9)** | 47 (22.3)***♦ [symbol] |
| −7 points | | | | |
| n (%) | | | | |
| Change ≦ | 8 (3.6) | 6 (5.9) | 15 (7.2) | 22 (10.4)** |
| −10 points | | | | |
| n (%) | | | | |

P-value based on CMH test
Significantly higher percentage of responders than placebo: *p ≦ 0.05; **p ≦ 0.01; ***p ≦ 0.001;
Significantly higher percentage of responders than 8 mg/day: ^{†}p ≦ 0.05; ^{‡}p ≦ 0.01;
Approached significance: ♦0.05 < p-value < 0.10;
The difference between 16 and 24 mg/day approached significance [symbol] : 0.05 <p-vlaue < 0.10.

An additional secondary indication captures overall changes in Activities of daily Living (ADL) performances as measured by the Alzheimer's Disease Cooperative Study Activities of Daily Living (ACDS/ADL) scale. As mentioned above this scale is comprised of 23 items that have been tested and validated in patients with mild to moderately severe Alzheimer's disease.

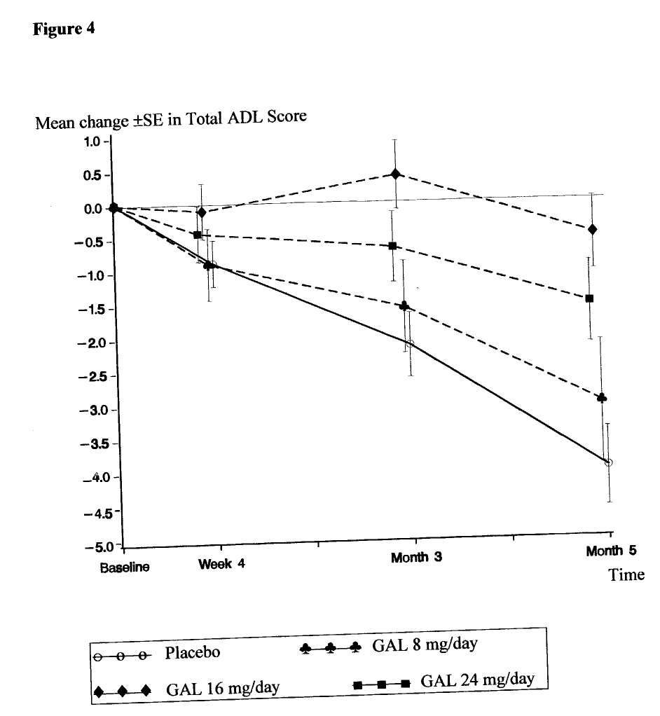

Galantamine treatment with 16 or 24 mg/day for 5 months was statistically more effective in maintaining the ADL total score at baseline levels than treatment with placebo or 8 mg/day of galantamine (Table 6). The dose-related effect of galantamine treatment is apparent in FIG. 4 that shows change of total ADL score over time.

TABLE 6
Change in Total ADL score from baseline at Month 5

| | Placebo | GAL 8 mg | GAL 16 mg | GAL 24 mg |
|---|---|---|---|---|
| Month | n = 235 | n = 106 | n = 212 | n = 212 |
| 5: (ob- | | | | |
| served | | | | |
| case) | | | | |
| Mean | −4.0 (0.59) | −3.1 (0.91) | −0.5 (0.55)***^{†} | −1.6 (0.61)** |
| (SE) | | | | |
| Month | n = 262 | n = 129 | n = 255 | n = 253 |
| 5: | | | | |
| (LOCF) | | | | |
| Mean | −3.8 (0.55) | −3.2 (0.79) | −0.7 (0.050)***^{‡} | −1.5 (0.56)** |
| (SE) | | | | |

Higher score indicates better condition. P-Values based on two-way ANOVA model.
Significantly more effective than placebo: **p ≦ 0.01; ***p ≦ 0.001
Significantly more effective than 8 mg/day: ^{†}p ≦ 0.05; ^{‡}p ≦ 0.01.

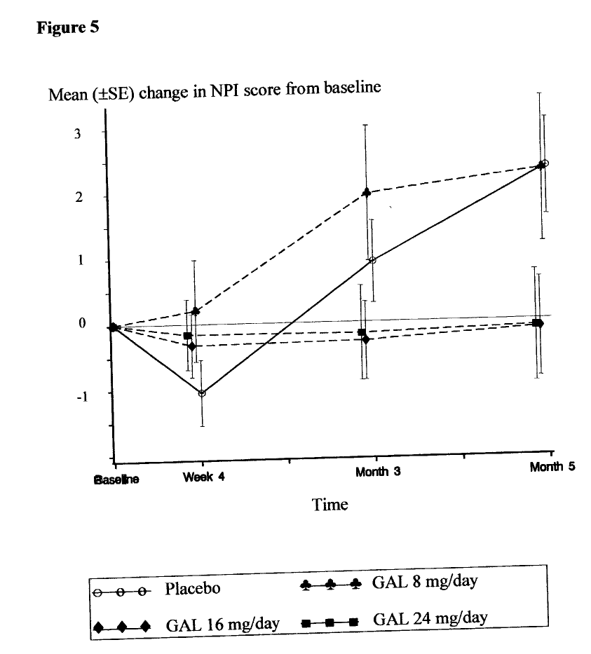

There was a statistically significant (p<0.05) benefit seen in the change in the total NPI score at Month 5 relative to baseline for 16 and 24 mg/day of galantamine compared with a deterioration with placebo or 8 mg/day of galantamine (Table 7 and FIG. 5). An increase in score indicated a worsening in condition. Thus, a maintenance of neuropsychiatric behaviour was observed with galantamine at 16 and 24 mg/day.

TABLE 7
Change in Total NPI score from baseline at Month 5

| | Placebo | GAL 8 mg | GAL 16 mg | GAL 24 mg |
|---|---|---|---|---|
| Month 5: | n = 234 | n = 106 | n = 211 | n = 212 |
| (observed case) | | | | |
| Mean (SE) | 2.3 (0.74) | 2.3 (1.121) | −0.1 (0.76)* | −0.1 (0.86)* |
| Month 5: (LOCF) | n = 262 | n = 129 | n = 255 | n = 253 |
| Mean (SE) | 2.0 (0.68) | 2.3 (1.00) | −0.1 (0.71)* | −0.0 (0.76)* |

Higher score indicates worsened condition. p-Values based on two-way ANOVA model.
Significantly more effective than placebo: *p ≦ 0.05

The most common adverse events were evenly distributed across treatment groups with the exception of events that are associated with cholinomimetic agents (Table 8). Of these related events, nausea, vomiting and anorexia showed a mild dose-related occurrence at a relatively low incidence.

TABLE 8
Incidence of most frequent (≧5%)
adverse events: number (%) of patients

| Adverse | | | | |
|---|---|---|---|---|
| event (Pre- | | | | |
| ferred term) | Placebo | GAL 8 mg | GAL 16 mg | GAL 24 mg |
| Total all | 286 | 140 | 279 | 273 |
| patients | | | | |
| Nausea | 13 (4.5%) | 8 (5.7%) | 37 (13.3%) | 45 (16.5%) |
| Vomiting | 4 (1.4%) | 5 (3.6%) | 17 (6.1%) | 27 (9.9%) |
| Anorexia | 9 (3.1%) | 8 (5.7%) | 18 (6.5%) | 24 (8.8%) |
| Agitation | 27 (9.4%) | 21 (15.0%) | 28 (10.0%) | 22 (8.1%) |
| Depression | 15 (5.2%) | 4 (2.9%) | 24 (8.6%) | 22 (8.%) |
| Urinary tract | 19 (6.6%) | 11 (7.9%) | 23 (8.2%) | 22 (8.1%) |
| infection | | | | |
| Dizziness | 10 (3.5%) | 7 (5.0%) | 15 (5.4%) | 19 (7.0%) |
| Injury | 12 (4.2%) | 5 (3.6%) | 12 (4.3%) | 16 (5.9%) |
| Diarrhea | 17 (5.9%) | 7 (5.0%) | 34 (12.2%) | 15 (5.5%) |
| Dyspepsia | 7 (2.4%) | 4 (2.9%) | 13 (4.7%) | 15 (5.5%) |
| Headache | 13 (4.5%) | 5 (3.6%) | 19 (6.8%) | 13 (4.8%0 |
| Weight | 4 (1.4%) | 2 (1.4%) | 15 (5.4%) | 13 (4.8%) |
| decrease | | | | |
| Fall | 14 (4.9%) | 11 (7.9%) | 14 (5.05%) | 12 (4.4%) |
| Rhinitis | 6 (2.1%) | 9 (6.4%) | 9 (3.2%) | 11 (4.0%) |
| Edema | 7 (2.4%) | 9 (6.4%) | 8 (2.9%) | 7 (2.6%) |
| peripheral | | | | |

For most adverse events of clinical interest, as shown in Table 9, there were either no differences or slight dose-related differences between treatment groups. For bradycardia, there was a higher incidence for patients treated with galantamine compared with placebo but there was no dose-related association apparent. For syncope, there was a slight dose-related increase in incidence with 24 mg/day of galantamine, however 3 of these cases occurred at a lower dose during titration, and are therefore attributable to a lower galantamine dose. Consequently, the incidences shown in Table 6 are very likely to be over-estimates for the occurrence of syncope at the higher doses. Furthermore, 10 of 18 patients who experienced a syncopal episode were taking concomitant cardiovascular medication including bata-blockers, calcium channel antagonists, ACE inhibitors, and/or diuretics. Of these 18 patients, 11 had active cardiovascular disease listed in their past medical history. Therefore, a majority of patients who experienced syncope had either a cardiovascular condition or were taking cardiovascular medication.

TABLE 9
Adverse events of clinical interest

| Adverse | | | | |
|---|---|---|---|---|
| event (Pre- | | | | |
| ferred term) | Placebo | GAL 8 mg | GAL 16 mg | GAL 24 mg |
| Total all | 286 | 140 | 279 | 273 |
| patients | | | | |
| Bradycardia | 1 (0.3%) | 5 (3.6%) | 7 (2.5%) | 8 (2.9%) |
| Convulsions | 2. (0.7%) | 0 | 0 | 1 (0.4%) |
| Fatigue | 6 (2.1%) | 3 (2.1%) | 10 (3.6%) | 13 (4.8%) |
| Muscle | 3 (1.0%) | 1 (0.7%) | 3 (1.1%) | 1 (0.4%) |
| weakness | | | | |
| Syncope | 2 (0.7%) | 2 (1.4%) | 5 (1.8%) | 9 (3.3%) |

The incidence of serious adverse events was comparable across all treatment groups and (with the exception of syncope) showed no dose-related trends (Table 10). The four most frequent serious adverse events with galantamine and with an incidence of at least 1% of patients in any group were injury, syncope, fall, and myocardial infarction. There were no dose-related increases in GI-related serious adverse events. The only serious adverse event that showed a dose-relationship was syncope, however, for reasons already provided, these rates may be an over-estimate.

TABLE 10
Serious adverse events (with ≧2 patients in any group)

| Adverse | | | | |
|---|---|---|---|---|
| event (Pre- | | | | |
| ferred term) | Placebo | GAL 8 mg | GAL 16 mg | GAL 24 mg |
| Total all | 286 | 140 | 279 | 273 |
| patients | | | | |
| Total patients | 31 (10.8%) | 14 (10.0%) | 28 (10.0%) | 35 (12.8%) |
| with any SAE | | | | |
| Injury | 4 (1.4%) | 1 (0.7%) | 1 (0.4%) | 5 (1.8%) |
| Syncope | 2 (0.7%) | 1 (0.7%) | 4 (1.4%) | 5 (1.8%) |
| Asthenia | 1 (0.3%) | 0 (0.0%) | 2 (0.7%) | 1 (0.4%) |
| Dyspnea | 1 (0.3%) | 0 (0.0%) | 2 (0.7%) | 3. (1.1%) |
| Pneumonia | 4 (1.4%) | 1 (0.7%) | 2 (0.7%) | 3 (1.1%) |
| Gi | 0 (0.0%) | 0 (0.0%) | 0 (0.0%) | 2 (0.7%) |
| haemorrhage | | | | |
| Vomiting | 1 (0.3%) | 0 (0.0%) | 2 (0.7%) | 1 (0.4%) |
| Abdominal | 1 (0.3%) | 0 (0.0%) | 2 (0.7%) | 0 (0.0%) |
| pain | | | | |
| Diarrhea | 1 (0.3%) | 0 (0.0%) | 3 (1.1%) | 0 (0.0%) |
| Nausea | 1 (0.3%) | 0 (0.0%) | 2 (0.7%) | 0 (0.0%) |
| Basal cell | 0 (0.0%) | 0 (0.0%) | 0 (0.0%) | 2 (0.7%) |
| carcinoma | | | | |
| Breast neo- | 2 (0.7%) | 0 (0.0%) | 0 (0.0%) | 0 (0.0%) |
| plasm female | | | | |
| Fall | 3 (1.0%) | 4 (2.9%) | 1 (0.4%) | 3 (1.1%) |
| Surgical | 1 (0.3%) | 0 (0.0%) | 3 (1.1%) | 0 (0.0%) |
| intervention | | | | |
| Thrombo- | 1 (0.3%) | 0 (0.0%) | 0 (0.0%) | 2 (0.7%) |
| phlebitis deep | | | | |
| Transient | 1 (0.3%) | 1 (0.7%) | 2 (0.7%) | 0 (0.0%) |
| ischemic attack | | | | |
| Myocardial | 2 (0.7%) | 3 (2.1%) | 1 (0.4%) | 1 (0.4%) |
| infarction | | | | |
| Agitation | 1 (0.3%) | 2 (1.4%) | 1 (0.4%) | 0 (0.0%) |
| Urinary tract | 0 (0.0%) | 1 (0.7%) | 2 (0.7%) | 0 (0.0%) |
| infection | | | | |
| Cardiac failure | 2 (0.7%) | 0 (0.0%) | 3 (1.1%) | 0 (0.0%) |
| Dehydration | 0 (0.0%) | 0 (0.0%) | 3 (1.1%) | 0 (0.0%) |
| Sepsis | 2 (0.7%) | 0 (0.0%) | 0 (0.0%) | 0 (0.0%) |

Throughout the trial there were 11 deaths. There was no apparent dose-related patterns in the occurrences of deaths. No death was considered by the investigator to be related to trail medication.

The results of this example confirmed that treatment with either 16 mg/day or 24/mg day of galantamine leads to statistically significant improvement in neuropsychiatric behaviour, as determined by the NPI score at month 5 relative to baseline with 16 mg/day and 24 mg/day compared with a deterioration with placebo or 8 mg/day of galantamine.

All scientific publications and patent documents are incorporated herein by reference.

The present invention has been described with regard to preferred embodiments. However, it will be obvious to persons skilled in the art that a number of variations and modifications can be made without departing from the scope of the invention as described in the following claims.

1. Use of an effective amount of galantamine or a pharmaceutically acceptable salt thereof for the production of a medicament for treating neuropsychiatric behaviour associated with Alzheimer's disease.

2. The use of claim 1 wherein the galantamine is administered from about 1 mg to about 100 mg per day.

3. The use of claim 2 wherein the galantamine is administered from about 5 mg to about 50 mg per day.

4. The use of claim 3 wherein the galantamine is administered from about 16 mg to about 32 mg per day.

5. The use of claim 4 wherein the galantamine is administered at a dosage of about 24 mg per day, wherein said dosage is reached after a titration of a dose of about 8 mg/day for about 1 week, followed by a dose of about 16 mg/day for about 1 week, followed by a daily dosage of about 24 mg/day thereafter.

6. The use of claim 4 wherein the galantamine is administered at a dosage of about 32 mg per day, wherein said dosage is reached after a titration of a dose of about 8 mg/day for about 1 week, followed by a dose of about 16 mg/day for about 1 week, followed by a dose of about 24 mg/day for about 1 week, followed by a daily dosage of about 32 mg/day thereafter.

7. The use of claim 4 wherein the galantamine is administered at a dosage of from about 16 mg to about 24 mg of galantamine, wherein said dosage is reached after a titration of from about 2 to about 10 weeks with the initial dose at about 8 mg increasing to a final dose of from about 16 mg to about 24 mg.

8. The use of claim 7 wherein the galantamine is administered at a dosage of about 24 mg/day, wherein said dosage is reached after a titration of a dose of about 8 mg/day for from about 2 weeks to about 4 weeks, followed by a dose of about 16 mg/day for from about 2 weeks to about 4 weeks, followed by the daily dosage of about 24 mg/day thereafter.

9. The use of claim 7 wherein the galantamine is administered at a dosage of about 24 mg/day, wherein said dosage is reached after a titration of a dose of about 8 mg/day for about 4 weeks, followed by a dose of about 16 mg/day for about 4 weeks, followed by the daily dosage of about 24 mg/day thereafter.

10. The use of claim 4 wherein the galantamine is administer at a dosage about 16 mg/day, wherein said dosage is reached after a titration of a dose of about 8 mg/day for from about 2 weeks to about 4 weeks, followed by the daily dosage of about 16 mg/day thereafter.

11. The use of claim 9 wherein the galantamine is administer at a dosage of about 16 mg/day, wherein said dosage is reached after a titration of a dose of about 8 mg/day for about 4 weeks, followed by the daily dosage of about 16 mg/day thereafter.
